(19) United States
(12) Patent Application Publication
(10) Pub. No.: US 2020/0246288 A1
(43) Pub. Date: Aug. 6, 2020

(54) OIL PALM PHENOLICS COMPOSITION FOR THE PROTECTION OF HUMANS, ORGANS, CELLS AND TISSUES AGAINST THE INJURIOUS EFFECTS OF EXPOSURE TO IONIZING RADIATION

(71) Applicant: MALAYSIAN PALM OIL BOARD, Kajang (MY)

(72) Inventors: Ravigadevi SAMBANTHAMURTHI, Kajang (MY); Yew Ai TAN, Kajang (MY); Vera Vladimirovna KOLEDOVA, Medford, MA (US); Robert Paul WEINBERG, Dixfield, ME (US); Cho Kyun RHA, Cambridge, MA (US); Anthony J. SINSKEY, Cambridge, MA (US)

(73) Assignee: MALAYSIAN PALM OIL BOARD, Kajang (MY)

(21) Appl. No.: 16/853,696

(22) Filed: Apr. 20, 2020

Related U.S. Application Data
(62) Division of application No. 16/067,130, filed on Jun. 29, 2018.

(30) Foreign Application Priority Data
Dec. 31, 2015 (MY) PI 2015704862

(51) Int. Cl. A61K 31/191; A61K 31/192; A61K 31/702; A61K 36/889; A61K 31/225; A61K 36/888

(52) U.S. Cl. CPC A61K 31/191; A61K 31/192; A61K 36/888; A61K 36/889; A61K 31/225; A61K 31/702

(57) ABSTRACT

A composition for preventing, treating, reversing, protecting against ameliorating the injuring effect of an ionizing radiation upon living cells, tissues, organs, and organisms, comprising oil palm phenolics (OOP) or vegetation liquor extract obtained from aqueous stream of a palm oil milling effluent is provided.

### FIELD OF INVENTION

The present invention relates generally to compositions for protection of living cells and tissues against exposure to radiation, and more particularly, the present invention relates to vegetation liquor or a botanical extract derived from vegetation liquor of the oil palm milling process, and more particularly for protection against the injurious biologic effects caused when cells are exposured to ionizing radiation. The botanical extract known as Oil Palm Phenolics (OPP) comprises phenolics as well as other bioactives including shikimic acid, fruit acids, fruit sugars, soluble fibre and vitamins.

### BACKGROUND OF THE INVENTION

Ionizing radiation is a form of radiant energy having substantial energy to displace electrons from their atomic or molecular orbital shells. Such radiation can be emitted in the form of solar radiation from sun; radon gas generated by the earth; beta, gamma radiations emitted by radioisotopes. However, such radiations can cause injury to living cells, and organisms as the radiations can damage and disrupt cellular functions at the biochemical level.

In another instance, the radiations can cause mutations within DNA, RNA. The radiations can also injure unborn fetus in utero. The radiations can also cause structural damage, in part through the formation of reactive free radicals, to DNA, RNA, proteins affecting various processes occurring within DNA, RNA, proteins, such as replication, mitosis, meiosis, transcription, translation, post translational modifications, signaling pathways, biosynthetic pathways, bio-degradative pathways, cellular pathways, and so on. However, such a harmful effects can be prevented, reduced, treated and/or ameliorated by a number of compositions.

Jin Dai and Russell J. Mumper (Molecules 2010, 15, 7313-7352) disclosed the extraction, analysis, and antioxidant and anticancer properties of phenolics obtained from a plant source. The paper also briefly mentions the effects of using phenolics in reducing the damage done by UV radiation (ionizing radiation).

Elrashid Saleh Mandi et al (Int J Nanomedicine. 2011; 6: 2499-2512) disclosed that Palm kernel oil esters (PKOEs)-based nanoemulsions loaded with P. urinaria extract for treatment of ageing of skin. Further, the study concludes that the ageing skin results from (ameliorating) the effects (affects) caused by ionizing radiation damage. However, Mandi et al don't disclose use of oil palm phenolics in treatment against radiation damage.

EP2155224 discloses cosmeceutical composition comprising phenolic compositions, fruit acids and sugars extracted from the vegetation liquor of the palm oil milling process which has been suggested. This composition is rich in antioxidants, and significantly improves skin health, including preventing aging of the skin.

Therefore, there exists a need for developing compositions which can prevent, reduce, treat and/or ameliorate the harmful effects of ionizing radiations upon living cells, tissues and organisms.

### SUMMARY OF THE INVENTION

In an aspect, a composition comprising oil palm phenolics (OPP) or vegetation liquor extracts obtained from aqueous stream of a palm oil mill effluents, which is provided for the protection of humans, organs, cells and tissues against the injurious effects of exposure to an ionizing radiation. The OPP or vegetation liquor extract includes at least one aqueous phytochemical extract, at lease one aqueous bioactive phenolic, and an acid and each of the aqueous phytochemical extract, bioactive compound and the acid singly, jointly, additively and/or synergistically protect against the injurious effects of the ionizing radiation.

In another aspect, a method of treatment against the injurious effects of the ionizing radiation is provided. The method includes administering a composition comprising OPP or vegetation liquor extracts obtained from aqueous stream of a palm oil mill effluents.

In yet another aspect, use of a composition comprising OPP against the injurious effects of the ionizing radiation on humans, cells, tissues and organs is provided.

### BRIEF DESCRIPTION OF DRAWINGS

Other objects, features, and advantages of the invention will be apparent from the following description when read with reference to the accompanying drawings. In the drawings, wherein like reference numerals denote corresponding parts throughout the several views:

FIG. 1 illustrates a comparison of prophylactic OPP exposure and therapeutic exposure, according to an illustrative embodiment of a present invention;

FIG. 2 illustrates a graph depicting prevention of radiation-induced lipid peroxidation by OPP, according to the illustrative embodiment of the present invention;

FIG. 3 illustrates a graph depicting prevention of specific polyphenols on radio-resistance HT-29 cells, according to the illustrative embodiment of the present invention; and

FIG. 4 illustrates a graph depicting comparison when radio-resistance HT-29 cells are treated with different preparations of OPP-derived components, according to the illustrative embodiments of the present invention.

### DETAILED DESCRIPTION OF THE PREFERRED EMBODIMENTS

Reference will now be made in detail to the preferred embodiments of the present invention, examples of which are illustrated in the accompanying drawings.

In the following detailed description, numerous specific details are set forth in order to provide a thorough understanding of the invention. However, it will be understood by those of ordinary skill in the art that the invention may be practiced without these specific details. In other instances, well known methods, procedures and/or components have not been described in detail so as not to obscure the invention.

The invention will be more clearly understood from the following description of the methods thereof, given by way of example only with reference to the accompanying drawings.

During the palm oil milling process, water-soluble bioactives are discarded in the waste stream. A recovery procedure for OPP has been developed to isolate concentrations suitable for biological applications from the aqueous waste stream i.e. vegetation liquor, such that it now provides an opportunity to transform a bioburden into a range of potential applications for health and wellness. The major constituents in the oil palm vegetation liquor extracts or OPP are phenolics, fruit acids, fruit sugars, soluble fibre and glycerol. In the instant case, OPP is used to develop compositions for treating radiation injury to humans, cells, tissues and organs by exposure to ionizing radiations such as X-ray radiations, Gamma-radiations or other such ionizing radiations.

Ionizing radiations have harmful effects on the living cells, tissue, and organisms. The present embodiment discloses chemical compositions, which can prevent, treat, or ameliorate the harmful effects of radiation exposure to living cells, tissue, and organisms. The chemical composition includes an aqueous phytochemical extract composition; an aqueous bioactive composition; and an acid. According to an embodiment, each of the aqueous compositions and the acid singly, jointly, additively and/or synergistically makes the living cells radioactive resistant or treat the damage by exposure to a radiation.

Radiation injury is damage to tissues caused by exposure to ionizing radiation. In the context of the present embodiment, the ionizing radiation refers to high-energy electromagnetic waves (ultraviolet, soft and hard x-rays and gamma rays) and particles ejected by radioactive nuclear decay (alpha particles, beta particles, and neutrons) that are capable of stripping electrons from atoms (ionization). By changing molecules in the highly ordered environment of the cell, ionizing radiation often disrupts and damages the cells. Cellular damage can cause illness, increase the risk of developing cancer, or both. Ionizing radiation is emitted by radioactive substances (radionuclides), such as uranium, radon, and plutonium. It is also produced by devices, such as x-ray and radiation therapy machines.

According to an embodiment, the composition for treating radiation injury includes a water-soluble bioactive botanical extract obtained from a plant Elaeis guineensis. Such an extract can also be referred to as oil palm phenolics or OPP or vegetation liquor extract. According to an embodiment, the bioactive compositions include but are not confined to three isomers of caffeoylshikimic acid, protocatechuic acid, and p-hydroxybenzoic acid. The bioactive composition may also include shikimic acid. Apart from the aqueous compositions and the acid, the composition may also include peptides, minerals, and oligosaccharides. Each of the compositions singly, jointly, additively and/or synergistically makes the living cells, tissues, and organisms radioactive resistant.

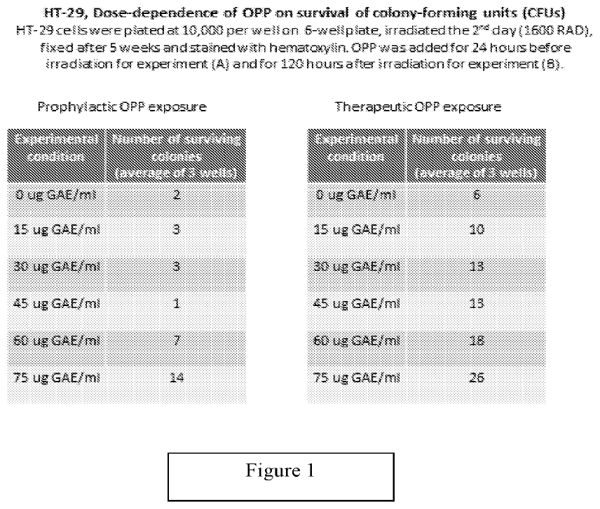

According to one of the present embodiment, prophylactically, there is significant protection against ionizing radiation at OPP concentrations of 65- and 75-μg GAE/ml. However, therapeutically, there is protection against ionizing radiation at OPP concentrations in the range of 30-to 75-μg GAE/ml. As per the prophylaxis experiment, HT-29 cells are plated on day 1. OPP is added on day 2 after 24 hours. The HT-29 cells are irradiated with 1600 Rads via a Cesium-137 gamma source, followed by changing the media. However, therapeutically, HT-29 cells are plated at day 1, followed by irradiation with 1600 Rads via a Cesium-137 gamma source after 48 hours. Thereafter, OPP is added to the HT-29 cells. At 168 hours, the media is changed. Such comparison of both types of exposures of OPP is shown in FIG. 1.

According to another embodiment, the OPP prevents, treats, reverses, protects against/or ameliorates the injuring effects of ionizing radiation on DNA. The OPP composition can prevent, treat, reverse, protect against/or ameliorate the injuring effects of ionizing radiation which affect the survival, viability and proliferation of living cells and tissues.

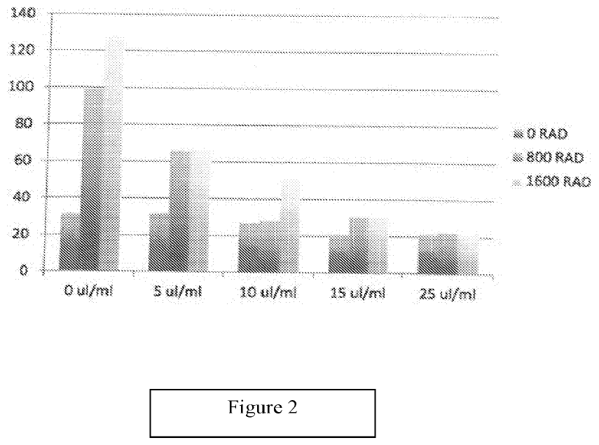

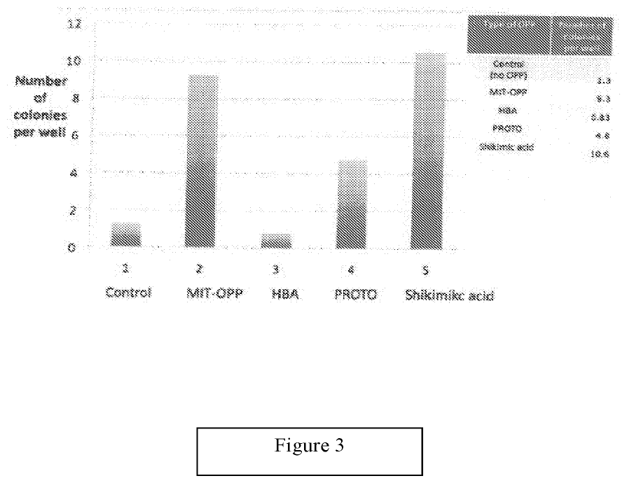

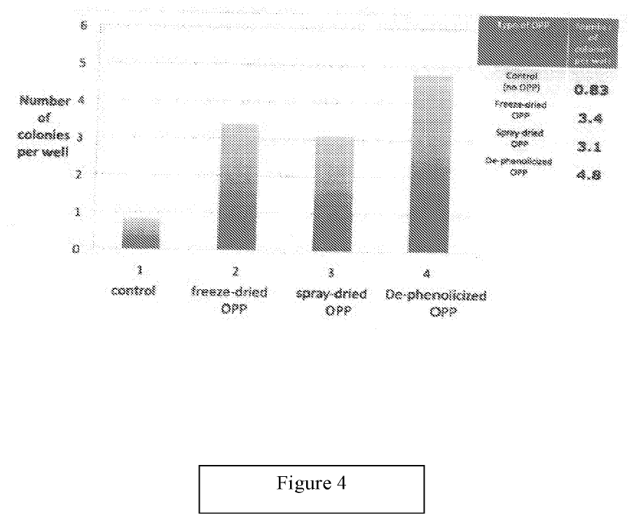

For instance, lipid peroxidation levels of HT-29 by TBAR assay is protected by OPP against 800 and 1600 rads HT-29, as illustrated in graph of FIG. 2. In another instance, as shown in FIG. 3, number of colonies abruptly increases in the presence of OPP and shikimic acid, thereby protecting HT-29 cells against gamma ionizing. In yet another instance shown in FIG. 4, HT-29 cells can be protected against ionizing radiation by different preparations of OPP such as freeze-dried, spray-dried, and vegetation liquor extract with major phenolics removed (de-phenolics). Out of the three preparations, number of colonies of HT-29 is highest in case of de-phenolics.

In an embodiment, a method of treating a subject, cell, tissues or organ from radiation injury caused by exposure to ionizing radiations is provided. The method includes administering to a subject a composition comprising OPP or vegetation liquor extract obtained from the aqueous stream of palm oil milling process. The composition may be administered topically. In an embodiment, the composition is administered orally.

The method of treatment comprising administering OPP to a subject may also prevent, treat, reverse, protect against/or ameliorate the injuring effects of ionizing radiation on DNA. The method may also prevent, treat, reverse, protect against/or ameliorate the—injurious biologic effects of ionizing radiation on living cells, tissues and organisms.

In an embodiment, a use of OPP or vegetation liquor extracts is provided. The use includes treating a subject against the injurious effects of radiation damage by ionizing radiation. The OPP may be used either topically or administered orally to treat the injurious effects of radiation damage by ionizing radiations. The use of OPP in treating radiation damage to a subject may also prevent, treat, reverse, protect against/or ameliorate the injuring effects of ionizing radiation on DNA. The use of OPP in treating radiation damage may also prevent, treat, reverse, protect against/or ameliorate the injuring effects of ionizing radiation on living cells, tissues, organs and organisms.

While the preferred embodiment of the present invention and its advantages has been disclosed in the above description, the invention is not limited there to but only by the scope of the appended claim.

As will be readily apparent to those skilled in the art, the present invention may easily be produced in other specific forms without departing from its essential characteristics. The present embodiments is, therefore, to be considered as merely illustrative and not restrictive, the scope of the invention being indicated by the claims rather than the foregoing description, and all changes which come within therefore intended to be embraced therein.

1. A composition for preventing, treating, reversing, protecting against or ameliorating the injuring effects of an ionizing radiation upon living cells, tissues, organs, and organisms, comprising vegetation liquor or a botanical extract derived from vegetation liquor of the oil palm milling process known as oil palm phenolics (OPP).

2. The composition of claim 1, wherein said OPP or vegetation liquor extract comprises an aqueous phytochemical extract, aqueous bioactive phenolics, soluble fibre and an acid; wherein, each of the aqueous phytochemical extract, bioactive compound and the acid singly, jointly, additively and/or synergistically protect against injuring effects of ionizing radiation.

3. The composition of claim 1, wherein the OPP or vegetation liquor extract is obtained from a plant Elaeis guineensis.

4. The composition of claim 2, wherein the bioactive phenolics include three isomers of caffeoylshikimic acid, protocatechuic acid, and p-hydroxybenzoic acid.

5. The composition of claim 1, wherein the composition includes shikimic acid.

6. The composition of claim 1, wherein the composition includes soluble fibre, peptides, minerals, and oligosaccharides.

7. The composition of claim 1, wherein the composition prevents, treats, reverses, protects against/or ameliorates the injuring effects of ionizing radiation on nucleic acids.

8. The composition of claim 6, wherein the injurious biologic effects of the ionizing radiation affects the survival, viability and/or proliferation of living cells, tissues, organs and organisms.

9. The composition of claim 1, wherein the composition prevents, treats, reverses, protects against/or ameliorates the injuring effects of ionizing radiation on lipids including glycolipids, phospholipids, and other lipid components of cells.

10. The composition of claim 1, wherein the composition prevents, treats, reverses, protects against/or ameliorates the injuring effects of ionizing radiation on cell membranes and membranous organelles.

11. The composition of claim 1, wherein the composition prevents, treats, reverses, protects against/or ameliorates the injuring effects of ionizing radiation on membranous organelles.

12. The composition of claim 1, wherein the composition prevents, treats, reverses, protects against/or ameliorates the injuring effects of ionizing radiation whereby reactive oxygen and reactive nitrogen species are formed by such ionizing radiation.
